(19) United States
(12) Patent Application Publication
(10) Pub. No.: US 2025/0359779 A1
(43) Pub. Date: Nov. 27, 2025

(54) SYSTEM AND METHOD FOR MOTION MEASUREMENT AND RECOVERY USING ARTIFICIAL INTELLIGENCE

(71) Applicant: arplo LLC, New Orleans, LA (US)

(72) Inventors: Victor Sonaike, New Orleans, LA (US); Michael Asevado, St. Bernard, LA (US); Jeff Orthober, SR., Egg Harbor, WI (US); Gabriel Hannon, New York, NY (US); Christopher Crouch, Mount Laurel, NJ (US); Llewellyn Dawkins, JR., Somerset, NJ (US); Yvette Susi Folse, Long Beach, MS (US); Bryan Soulie, Fort Lauderdale, FL (US); Moodlagiri Pai, Euless, TX (US)

(73) Assignee: arplo LLC, New Orleans, LA (US)

(21) Appl. No.: 18/674,237

(22) Filed: May 24, 2024

(51) Int. Cl. A61B 5/11; G06T 7/246; G06V 40/16; G06V 40/20

(52) U.S. Cl. CPC A61B 5/1114; A61B 5/1121; A61B 5/1123; A61B 5/1128; A61B 5/7264; G06T 7/246; G06V 40/161; G06V 40/168; G16H 20/30; G06T 2207/20044; G06V 2201/07

(57) ABSTRACT

A system and a method for motion measurement and recovery using artificial intelligence is provided. The system receives user data associated with a first user. The user data comprises demographic data, medication data, and historical data. The system receives video data indicative of an activity performed by the first user. The system determines anatomical data associated with the first user based on processing of the video data. The anatomical data includes joints data for one or more anatomical joints of the first user and movement data associated with each of the one or more anatomical joints of the first user. The system generates, using an artificial intelligence (AI) model, a score for the activity performed by the first user based on the user data and the anatomical data. Additionally disclosed the system and method for motion measurement and recover using artificial intelligence utilizes marker-less motion technology for capturing movement data.

### TECHNOLOGICAL FIELD OF THE INVENTION

The present invention pertains to rehabilitation of patients after motor impairments and more specifically to a system and a method for motion measurement and recovery of patients using artificial intelligence.

### BACKGROUND OF THE INVENTION

Motor rehabilitation is a process of restoring physical function and mobility in individuals with movement impairments. Conventionally, an individual (such as, a patient) with movement impairment needs to perform several movements (for example, exercises) for effective recovery. For example, healthcare professionals (such as, a therapist or a physician) may advise specific exercises to the individual based on a type of movement impairment. However, such a therapist or physician might not be able to dedicate adequate time to the individual during clinic sessions, thereby leading to assigning homework exercises to the individual. This may pose a challenge for the individual to maintain interest and accurately repeat the required exercises, thereby making compliance difficult and causing them to struggle. Moreover, with the growing population, there is a need for an increase in demand for healthcare professionals. Such a shortage of healthcare professionals worsens the situation. Additionally, economic constraints further limit the number of therapy sessions covered by insurance, thereby hindering the achievement of optimal outcomes.

Therefore, there is a need to develop effective solutions to optimize rehabilitation of patients after motor impairments and patient adherence.

### DESCRIPTION OF THE RELATED ART

Individuals suffering from movement impairments often experience hardships in regard to sustainably performing exercises to effectively recover from such movement impairments. Having the ability to be provided with assistance and guidance in real-time during such exercises can potentially help speed up recovery time for these individuals. The additional ability to obtain recordings of each exercise session over time provides an advantage to the therapist or physician, as the therapist and/or physician's access to such recordings over time can help the healthcare professional provide adjustments and changes to the exercise regime more efficiently and effectively. It is the goal of the present invention to utilize the benefits of the recordings and real-time assistance provided by Artificial Intelligence, without the need for a wearable device attached to the patient, to assist patients suffering from movement impairments recovery quicker while providing healthcare professionals with additional and more accurate data on each patient's progress thereby aiding in providing more effective and efficient treatment plans.

U.S. Pat. No. 11,672,477 B2 discloses devices, systems, and methods for monitoring musculoskeletal (MSK) health conditions of an individual, including joint flexibility, strength, and endurance as part of a patient's overall care plan. The system and methods require the use of sensors worn anywhere on the human body, utilizing a mobile application, implementing software-based analytics, and a care management engine running on a cloud-computing infrastructure.

WIPO International Application Publication No. WO 2022/217360 A1 discloses a diagnostic platform that is able to apply machine learning models to images generated by consumer computing devices to generate outputs that can be used to ascertain the health state of individuals. The diagnostic platform obtains and then examines a standardized set of images, for example, of different anatomical regions as part of a disease detection system that applies products supported by artificial intelligence to improve the accessibility of healthcare services. The platform generates an interface that prompts the patient and instructs the patient to generate images of different anatomical regions of the body, for example, images of the face, eyes, and tongue. Then, the platform generates an interface that includes a summary of the analysis of the images, which, specifies whether the platform discovered any features in the images that are indicative of a disease and providing recommendations for improving the health state of the patient.

U.S. Pat. No. 11,324,439 B2 discloses a method that includes (1) receiving images of a subject and (2) a total mass value for the subject, a first machine learning model is executed to identify joints of the subject, a second machine learning model is executed to determine limbs of the subject based on the joints and the images, and generating a three-dimensional (3D) representations of a skeleton based on the joints and the limbs of the subject.

U.S. Patent Application Publication No. US 2023/0170076 A1 discloses a system and method related to predicting, using adaptive artificial intelligence techniques, typical and aberrant physiological reactions of a patient to psychiatric counseling. Treatment plans are determined and calculated based on previously gathered demographic and/or biometric data, and/or modifications to treatment plans are determined and/or implemented based on emergent recognition of reaction types, such as reclassifying reactions that would previously have been deemed typical as aberrant (or vice versa). Patient behavior during counseling sessions are identified as typical or aberrant. Demographic, biometric, and time-based information are collected and processed using a machine learning task, analytics, and/or “big data,” to predict the best next steps for a patient with a mental illness that is under treatment by a professional counselor. The biometric data incorporated in a predictive model of patient behavior is collected, using video streams, voice streams, wearable devices, hand-held devices, smart phones, and other devices/techniques.

U.S. Patent Application Publication No. US 2021/0335478 A1 discloses a system and method for developing a treatment plan using multi-stage machine learning, including identifying at least one cognitive distortion of a user by applying a first machine learning model created from extracted data related to the user. By applying a second machine learning model created from extracted data related to the user and to the output of the first machine learning model, where the second machine learning model is a task recommender model trained using training cognitive distortions and the training user-created content, to generate a treatment plan including digital therapeutics exercise tasks for the user and includes additional aspects of treatment including, but not limited to, prescribing group therapy, formal and informal peer support, prescribing virtual reality sessions, prescribing pharmaceuticals, or a combination thereof in tandem with the digital therapeutics.

U.S. Pat. No. 9,861,300 B2 discloses an interactive virtual care system including a user sensory module to acquire multi-modal user data related to user movement, a data analysis module to compare the multi-modal user data to predetermined historical user data and/or statistical norm data for users is used to identify an anomaly in the user movement. The method includes face-to-face video and two-way sharing of a patient's health information, the use of computer-vision technology to assist remote interaction by capturing and analyzing the patient's movements, and further includes computer-assisted speech and audio analysis of a healthcare provider's and patient's interactions. The remote responder sends exercises to the user interface module for user diagnosis and evaluation, otherwise communicate with the user via the responder sensory module, the user performs the exercises or proceeds as directed by the remote responder, and all user activities are captured by the user sensory module.

U.S. Pat. No. 10,271,776 B2 discloses a method for analyzing and monitoring mobility abnormalities of human patients including the following stages: 1) capturing a physiotherapeutic sequence of a scene that includes 3D positioning and orientations of the body parts of the patient over time; 2) monitoring, over a physiotherapeutic session, a set of key points on the patient while the patient performs physiotherapeutic exercises from a set of predefined sequences of body-related and limb-related postures and gestures; and 3) analyzing the monitored set of key points during the physiotherapeutic session, to yield an assessment of the level of compliance of the patient in performance of the physical training or physiotherapeutic exercises, based at least partially on an abnormality mobility profile. The system and method, requires the use of a calibration system that includes one or more sensors configured to capture 3D positioning and orientation of the limbs of the patient.

U.S. Pat. No. 10,332,631 B2 discloses an integrated medical platform system for automated medical decision-making, including a first parser configured to parse text associated with medical information sources to obtain medical information and a second parser configured to parse patient data to obtain processed patient data. The first parser and the second parser are configured to structure the medical information to form structured medical metadata in an intelligent medical database. The integrated medical platform assists, particularly in an online environment, a patient or attending physician in determining possible diagnoses with accompanying statistical likelihoods, complete with recommended treatment and patient management plans, as well as, facilitating self-monitoring and management of chronic health conditions of the patient. The method provides, to a patient or attending physician, a set of possible diagnoses with accompanying statistical likelihoods, complete with recommended treatment, patient management plans, and the ability to project and/or track the treatment prognosis of a patient.

U.S. Pat. No. 9,536,052 B2 discloses a clinical predictive and monitoring system that includes a data store operable to receive and store data associated with a database of patients selected from medical and health data, including additional data of a number of social, behavioral, lifestyle, and economic data. A predictive model is used to identify at least one high-risk patient associated with a medical condition, a risk logic module that applies the predictive model to the patient data is used to determine a risk score associated the medical condition and identifies at least one high-risk patient. The variety of data is used to determine a disease risk score for selected patients so that they may receive more targeted intervention, treatment, and care that is better tailored and customized to their individual conditions and needs.

Chinese Patent Office Application Publication No. CN113409913A discloses according to the machine translation, an assessment method capable of judging the recovery stage of upper limb motor functions of a patient with paraplegia after stroke when a rehabilitation doctor and a therapist are absent by utilizing bone tracking technology and depth image data of the Microsoft Kinect 2.0 and combining function assessment technology based on a Brunnstrom upper limb motor function assessment table.

All aforementioned patents and publications are incorporated herein by reference.

While these devices and methods may be suitable for the particular purposes employed, they would not be as suitable, or suitable at all, for the purposes of the present invention as disclosed hereafter.

While the prior art discloses various devices, apparatuses, and methods for collecting patient data, movement data of a patient, and improving treatment plans, the present invention implements the use of markerless motion capture technology without the need for wearable devices or specialized camera devices in combination with artificial intelligence to assist patients and healthcare providers in real-time to more effectively and efficiently perform exercise treatment plans for patients suffering from motor impairments. Additionally, the invention disclosed herein provides the advantage of providing healthcare professionals with aggregated data based upon captured patient movement data, health records, and demographic data, to assist in creating more personalized treatment plans for aiding in more effective patient recovery.

### SUMMARY OF THE INVENTION

It is one prospect of the present invention to provide a system and method that focuses on motion measurement and recovery of patients after motor impairments using artificial intelligence.

In one aspect, a system for motion measurement and recovery using artificial intelligence is provided. The system includes one or more processors and a memory coupled to one or more processors. The one or more processors are configured to receive user data associated with a first user. The user data includes demographic data, medication data, and historical data. Further, the one or more processors are configured to receive video data indicative of an activity performed by the first user. The one or more processors are further configured to determine an anatomical data associated with the first user based on processing of the video data. The anatomical data includes joints data for one or more anatomical joints of the first user and a movement data associated with each of the one or more anatomical joints of the first user. Thereafter, the one or more processors are further configured to generate, using an artificial intelligence (AI) model, a score for the activity performed by the first user based on the user data, and the anatomical data.

In additional system embodiments, the joints data for the one or more anatomical joints of the first user includes location data associated with each of the one or more anatomical joints, and an angle data associated with each of the one or more anatomical joints.

In additional system embodiments, the movement data associated with each of the one or more anatomical joints of the first user includes a drift value associated with a movement of a first anatomical joint, an angular degree value associated with the movement of the first anatomical joint, a velocity value associated with the movement of the first anatomical joint, a strength value associated with the movement of the first anatomical joint and a stability value associated with the movement.

In other system embodiments, the processing of the video data further includes a sequential execution of image preprocessing operation, and marker-less motion capture operation on the video data.

In additional system embodiments, the one or more processors are further configured to determine marker-less motion data based on the processing of the video data. Further, one or more processors are configured to one or more anatomical landmarks associated with the first user based on the marker-less motion data. Thereafter, one or more processors are configured to determine the anatomical data based on the identified one or more anatomical landmarks associated with the first user.

In other additional system embodiments, the anatomical data may further include limb parameter data associated with the first user.

In additional system embodiments, the one or more processors are configured to render a set of instructions associated with the activity to be performed by the first user, and determine the anatomical data based on the set of instructions.

In additional system embodiments, the one or more processors may be further configured to receive a user input associated with the first user and modify the set of instructions associated with the activity to be performed by the first user based on the received user input.

In other additional system embodiments, the AI model may be further configured to generate the set of instructions based on the received user data.

In additional system embodiments, the one or more processors are further configured to generate an output indicative of a modification of a movement of at least the first anatomical joint of the one or more anatomical joints based on the generated score.

In other additional system embodiments, the one or more processors are further configured to transmit the generated score to a second user. Further, the one or more processors are configured to receive a user input associated with the second user and update the generated output based on the received user input.

In additional system embodiments, the one or more processors are further configured to generate a report associated with the first user. The report includes at least the user data, the anatomical data, and the generated score, and determines a recovery level of the first user based on the generated report.

In additional system embodiments, the one or more processors are further configured to receive at least a first image of an environment from an image capturing device. Further, the one or more processors are configured to determine the presence of the first user in the environment based on the received first image. Thereafter, the one or more processors are further configured to render the activity to be performed by the first user and obtain the video data associated with the rendered activity to be performed by the first user.

In other additional system embodiments, the one or more processors are configured to determine the number of times the activity is performed by the first user based on the video data. Further, the one or more processors are configured to determine a range of motion associated with at least a first anatomical joint of the one or more anatomical joints based on the determined anatomical data. Thereafter, the one or more processors are configured to generate the score associated with at least the first anatomical joint for the activity performed by the first user based on a number of times the activity is performed and the range of motion.

In yet other additional system embodiments, the generated score further includes a drift score associated with at least a first anatomical joint of the one or more anatomical joints, a stability score associated with at least the first anatomical joint of the one or more anatomical joints, a strength score associated with at least the first anatomical joint of the one or more anatomical joints, and a range score associated with at least the first anatomical joint of the one or more anatomical joints.

In additional system embodiments, the one or more processors are configured to detect a facial region of the first user based on the processing of the video data. Further, the one or more processors are configured to determine one or more facial features associated with the first user based on the detected facial region. Thereafter, the one or more processors are configured to compare at least a first feature from the one or more facial features and a corresponding reference facial feature from one or more reference facial features and render a set of instructions associated with the activity to be performed by the first user based on the comparison.

In other additional system embodiments, the user data further includes historical anatomical data associated with the first user. The one or more processors are configured to compare the historical anatomical data with the determined anatomical data. Further, the one or more processors are configured to generate the score for the activity performed by the first user based on the comparison.

In another aspect, a system for motion measurement is provided. The system includes one or more processors and a memory coupled to the one or more processors. The one or more processors are further configured to receive video data indicative of an activity performed by the first user. The one or more processors are further configured to determine anatomical data associated with the first user based on processing of the video data. The anatomical data includes a joints data for one or more anatomical joints of the first user and a movement data associated with each of the one or more anatomical joints of the first user. The one or more processors are further configured to compare the anatomical data associated with the first user and a historical anatomical data. Thereafter, the one or more processors are configured to generate a score associated with the activity performed by the first user based on the comparison.

In additional system embodiments, the one or more processors are further configured to determine marker-less motion data based on the processing of the video data. Further, the one or more processors are configured to identify one or more anatomical landmarks associated with the first user based on the marker-less motion data. Thereafter, the one or more processors are configured to determine the anatomical data based on the identified one or more anatomical landmarks associated with the first user.

In yet another aspect, a method for motion measurement and recovery using artificial intelligence is provided. The method includes a first step of receiving user data associated with a first user. The user data includes a demographic data, a medication data, and a historical data. The method includes a second step of receiving a video data indicative of an activity performed by the first user. The method includes a third step of determining an anatomical data associated with the first user based on processing of the video data, wherein the anatomical data includes a joints data for one or more anatomical joints of the first user and a movement data associated with each of the one or more anatomical joints of the first user. The method includes a fourth step of generating, using an artificial intelligence (AI) model, a score for the activity performed by the first user based on the user data, and the anatomical data.

### BRIEF DESCRIPTION OF THE DRAWINGS

Having thus described example embodiments of the invention in general terms, illustrative embodiments of the present invention are described herein with reference to the accompanying drawings, which are not necessarily drawn to scale, and wherein:

FIG. 1 is a diagram that illustrates a network environment within which a system is implemented, in accordance with an embodiment of the present invention;

FIG. 2 illustrates a block diagram of the system of FIG. 1, in accordance with an embodiment of the present invention;

FIG. 3 is a diagram that illustrates exemplary operations for motion measurement and recovery of patients using artificial intelligence, in accordance with an embodiment of the present invention;

FIG. 4 is a diagram that illustrates exemplary operations for marker-less motion measurement of the patients, in accordance with an embodiment of the present invention;

FIG. 5A and FIG. 5B collectively are diagrams that illustrate exemplary user interfaces for measurement and recovery of the patients, in accordance with an embodiment of the present invention; and

FIG. 6 is a flowchart that illustrates an exemplary method for motion measurement and recovery of the patients, in accordance with an embodiment of the present invention.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Additionally, reference in this specification to “Activity”, “Movement”, or “Exercise” should be understood as interchangeable by one of ordinary skill in the art. Moreover, various features are described which may be exhibited by some embodiments and not by others. Similarly, various requirements are described which may be requirements for some embodiments but not for other embodiments.

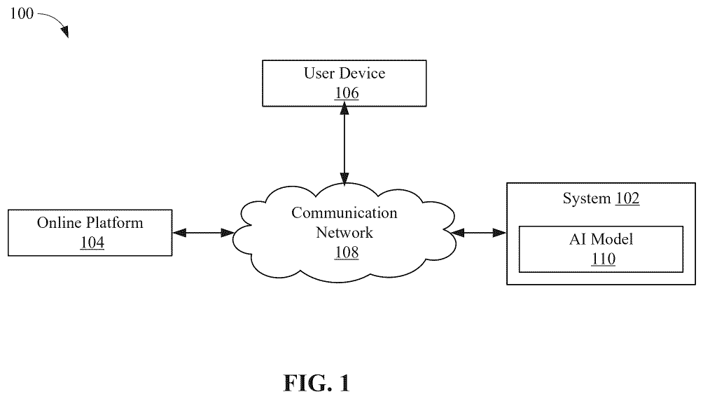

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Turning now to FIG. 1-FIG. 6, a brief description concerning the various components of the present disclosure will now be briefly discussed. Reference will be made to the figures showing various embodiments of a system and a method for motion measurement and recovery of patients.

FIG. 1 is a diagram that illustrates a network environment 100 within which a system is implemented, in accordance with an embodiment of the disclosure. The network environment 100 includes a system 102, an online platform 104, a user device 106, and a communication network 108. For example, the online platform 104 may be associated with a telemedicine platform, a healthcare website, or application, and so forth.

Typically, a user creates an account on the online platform 104 to access the services of the online platform 104. For example, the user, such as a patient, builds a profile on the online platform 104, using an interactive web form available on the online platform 104. The interactive web form may require user data, such as, but not limited to, demographic information associated with the user, medication information associated with the user, and historical medication information associated with the user, to build a personalized user profile.

The user device 106 includes suitable logic, circuitry, and/or interfaces that may be designed for a specific task within the network environment 100. The user device 106 plays a crucial role in receiving requests from the user, processing data, and delivering the data efficiently. The user device 106 may be designed for high-performance computing and data handling, ensuring that the user requests are handled accordingly and that the requested content is delivered to the user seamlessly. For example, the user device 106 includes but is not limited to, a computer, a laptop, a smartphone, or a tablet.

The system 102 includes suitable logic, circuitry, interfaces, and/or code that are configured to optimize motion measurement of users and rehabilitation of users (for example, patients) after motion impairments. The system 102 is equipped with a high-speed network interface, a multi-core processor, and a memory, the hardware configuration supports real-time image processing and analysis. The custom software orchestrates the communication network 104 monitoring process. The system 102 analyzes the motion impairments and leverages the use of artificial intelligence (AI) for efficient motion measurement and rehabilitation of the users. The system 102 further provides image processing, data analysis, and real-time monitoring.

The system 102 further includes an AI model 110. The AI model 110 may correspond to a neural network-based classifier. The neural network may be a computational network or a system of artificial neurons, arranged in a plurality of layers, as nodes. The plurality of layers of the neural network may include an input layer, one or more hidden layers, and an output layer. Each layer of the plurality of layers may include one or more nodes (or artificial neurons). Outputs of all nodes in the input layer are coupled to at least one node of the hidden layer(s). Similarly, inputs of each hidden layer may be coupled to outputs of at least one node in other layers of the neural network. Outputs of each hidden layer are coupled to inputs of at least one node in other layers of the neural network. Node(s) in the final layer receive inputs from at least one hidden layer to output a result.

The number of layers and the number of nodes in each layer may be determined from hyper-parameters of the neural network. Such hyper-parameters may be set before or while training the neural network on a training dataset. Each node of the neural network corresponds to a mathematical function (e.g., a sigmoid function or a rectified linear unit) with a set of parameters, tunable during training of the neural network. The set of parameters may include, for example, a weight parameter, a regularization parameter, and the like. Each node uses the mathematical function to compute an output based on one or more inputs from nodes in other layer(s) (e.g., previous layer(s)) of the neural network. All or some of the nodes of the neural network correspond to the same or a different mathematical function.

In the training of the neural network, one or more parameters of each node of the neural network are updated based on whether an output of the final layer for a given input (from a training dataset) matches a correct result based on a loss function for the neural network. The above process may be repeated for the same or a different input until a minimum loss function is achieved, and a training error is minimized. Several methods for training are known in the art, for example, gradient descent, stochastic gradient descent, batch gradient descent, gradient boost, meta-heuristics, and the like.

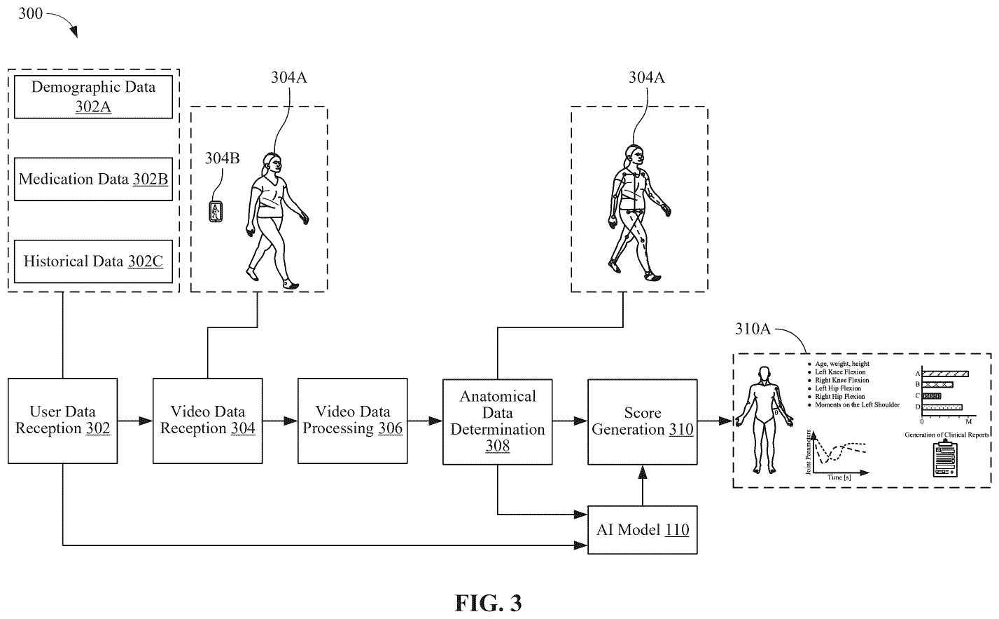

The neural network may include electronic data, such as, for example, a software program, code of the software program, libraries, applications, scripts, or other logic or instructions for execution by a processing device, such as circuitry. The neural network is implemented using hardware including a processor, a microprocessor (e.g., to perform or control the performance of one or more operations), a field-programmable gate array (FPGA), or an application-specific integrated circuit (ASIC). Alternatively, in some embodiments, the neural network may be implemented using a combination of hardware and software. Although in FIG. 1, the AI model 110 is shown integrated within the system 102, the disclosure is not so limited. Accordingly, in some embodiments, the AI model 110 may be a separate entity in the system 102, without deviation from the scope of the disclosure. Examples of the AI model 110 may include, but are not limited to, an artificial neural network (ANN) model, a deep neural network (DNN) model, a convolutional neural network (CNN) model, a fully connected neural network, and/or a combination of such networks. Details about the AI model 110 are provided, for example, in FIG. 3.

In one embodiment, the system 102 is communicatively coupled to the online platform 104, the user device 106, or any other device, via a communication network 108. The communication network 108 may be wired, wireless, or any combination of wired and wireless communication networks, such as cellular, Wi-Fi, internet, local area networks, or the like. In some embodiments, the communication network 108 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks (for e.g. LTE-Advanced Pro), 5G New Radio networks, ITU-IMT 2020 networks, code division multiple access (CDMA), wideband code division multiple access (WCDMA), wireless fidelity (Wi-Fi), wireless LAN (WLAN), Bluetooth, Internet Protocol (IP) data casting, satellite, mobile ad-hoc network (MANET), and the like, or any combination thereof.

All the components in the network environment 100 may be coupled directly or indirectly to the communication network 108. The components described in the network environment 100 may be further broken down into more than one component and/or combined in any suitable arrangement. Further, one or more components may be rearranged, changed, added, and/or removed.

In operation, the system 102 is configured to receive a user data associated with a first user. In an example, the first user may correspond to a patient with movement impairment. Such movement impairment may be due to heart stroke, neurological injury, orthopedic injury or any other physically debilitating diseases or injury, for example, but not limited to rotator cuff tear, frozen shoulder, and the like. The user data may include, but is not limited to, demographic information, medication information, and historical medication information associated with the first user. Details associated with the user data are provided for example, in FIG. 3.

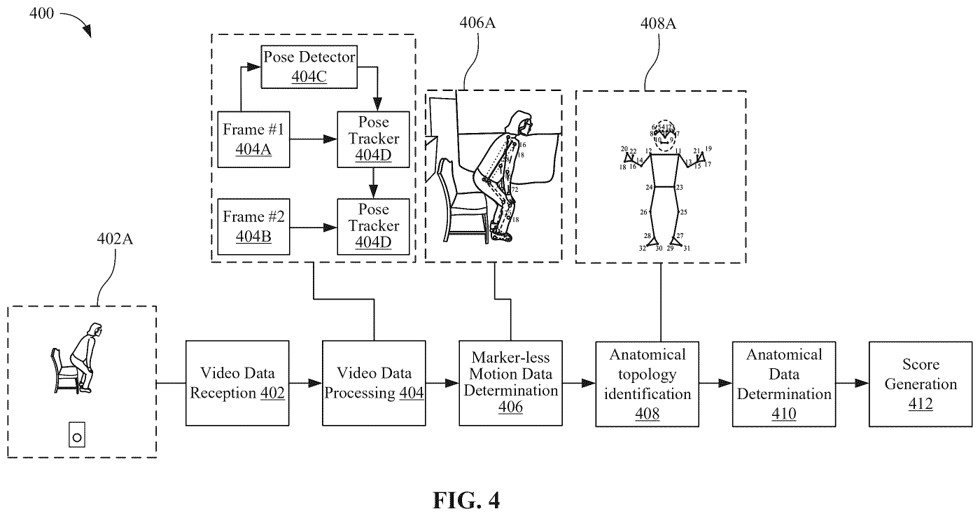

Further, the system 102 is configured to receive video data indicative of an activity performed by the first user. The activity performed by the first user corresponds to various movements or specific exercises performed by the first user. In an embodiment, the system 102 is configured to render a set of instructions associated with the activity to be performed by the first user. The set of instructions includes but are not limited to arm raise, arm lean, touch hands behind back, stand up-sit down, leg raise, and arm curl. Thereafter, the system 102 is configured to determine an anatomical data associated with the first user based on processing of the video data. Details associated with the processing of the video data for determination of the anatomical data are further provided, for example, in FIG. 3 and FIG. 4.

The anatomical data corresponds to information associated with the structure and configuration of a human body (for example, the first user). In an example, the anatomical data includes anatomical key points that correspond to key landmarks on the human body to identify structure of the body. Such anatomical key points on the body include but are not limited to joints, bones, muscles, and other anatomical features. Further, the system 102 is configured to analyze such anatomical key points to estimate a pose of the first user. The estimation may be based on one or more image processing algorithms, such as object detection algorithms, deep learning algorithms, and other methods which are known to one ordinarily skilled in the art.

In another embodiment, the anatomical data includes but is not limited to a joints data for one or more anatomical joints of the first user, and a movement data associated with the each of the one or more anatomical joints of the first user. The one or more anatomical joints of the first user corresponds to a joint where two or more bones meet in the human body, thereby allowing movement and providing mechanical support to the human body. The one or more anatomical joints of the first user includes but are not limited to fibrous joint, cartilaginous joint, and synovial joints.

Further, the joints data for the one or more anatomical joints of the first user include location data associated with each of the one or more anatomical joints, and an angle data associated with each of the one or more anatomical joints. The location data associated with each of the one or more anatomical joints of the first user may refer to a position of the joint, such as where the two or more bones meet in the human body. Further, the angle data associated with each of the one or more anatomical joints refers to an angle associated with the joint of the human body to measure relative orientation between two adjacent bones forming the joint. Additionally, the system 102 is configured to analyze the angle data to identify a degree of flexion, extension, or rotation occurring at the joint.

The movement data associated with each of the one or more anatomical joints of the first user further includes a drift value associated with a movement of a first anatomical joint, an angular degree value associated with the movement of the first anatomical joint, a velocity value associated with the movement of the first anatomical joint, a strength value associated with the movement of the first anatomical joint and a stability value associated with the movement. The drift value associated with the movement of a first anatomical joint refers to a degree of deviation in the position of the joint over time due to prolonged or repetitive movements. Such deviation occurs due to muscle fatigue, ligament laxity, joint instability, or biomechanical imbalance. The angular degree value associated with the movement of the first anatomical joint refers to a maximum or a minimum angle value associated with the movement of the first anatomical joint. The velocity value associated with the movement of the first anatomical joint refers to a rate at which the position of joints changes over time. In other words, the speed at which the joint moves from one position to another position. The velocity value includes but is not limited to angular velocity for rotational movements, linear velocity for translational movements. The strength value associated with the movement of the first anatomical joint refers to force (or torque) that joint produces or withstands during the movement. The strength value may be associated with muscles, ligaments, tendons, and other supporting structures surrounding the joints. The stability value associated with the movement refers to the ability of the joint to maintain its position during various activities. For example, the stability value includes, but is not limited to an inherent static stability of the joint, and a dynamic stability of the joint during the movement.

In yet another embodiment, the anatomical data further includes a limb parameter data associated with the first user. The limb parameter data associated with the first user refers to dimensional data associated with a limb of the first user for example, size, shape, and proportions of the limbs. The limb parameter data associated with the first user includes, but is not limited to length of the limb, segment mass of the limb, circumference of the limb, joint range of motion (ROM) of the limb, muscle cross-sectional area (CSA) of the limb, joint width of the limb, and joint circumference of the limb. Details associated with the determination of the anatomical data are provided, for example, in FIG. 3 and FIG. 4.

In another embodiment, the system 102 is configured to analyze the retrieved user data and the determined anatomical data, thereby providing valuable information associated with the first user. Such information may be employed to determine a type of orthopedic care required by the first user. Further, the system 102 is configured to transmit such information to a second user (such as, a healthcare worker/provider). The healthcare worker determines personalized treatment plans, and medications for the first user based on the information, thereby optimizing patient care, and treatment outcomes. Further, the system 102 leverages the use of the AI model 110 to provide suggestions for the personalized treatment plans, and medications to improve the recovery process of the patient.

Furthermore, the system 102 retrieves and analyzes data associated with patient recovery and rehabilitation plans, thereby acting as a telemedicine pipeline among the patients and the healthcare workers. This allows the system 102 to track, assess, and recommend improvements for patient recovery and optimum treatment plans for stroke victims and potentially other ailments. Additionally, the system 102 leverages the use of the AI model 110 to determine the most effective treatment plans for patient recovery when compared to demographic information.

Additionally, fewer, or different components may be provided in the network environment 100. For example, a server, a router, a database, additional computers or workstations, administrative components, such as an administrative workstation, a gateway device, and network interfaces may be provided. While the components in FIG. 1 are shown as separate from one another, one or more of these components may be combined. In this regard, a processor of the system 102 may be communicatively coupled to the components shown in FIG. 1 to carry out the desired operations and wherever required modifications may be possible within the scope of the present disclosure.

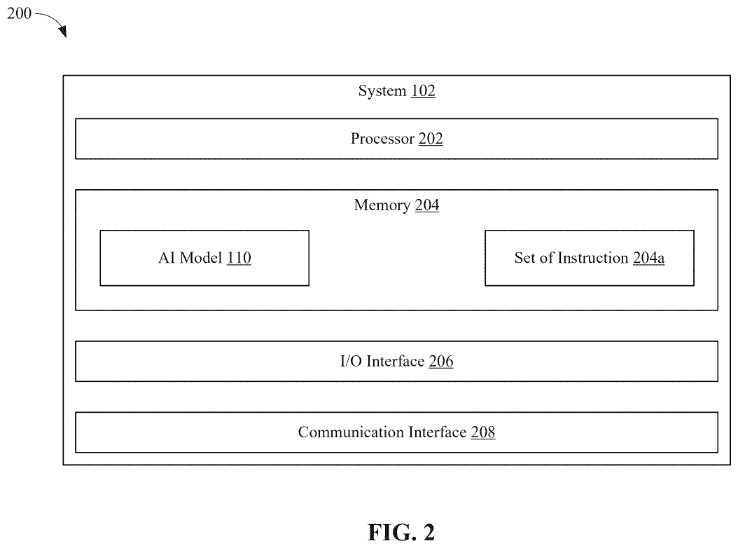

FIG. 2 illustrates a block diagram of the system of FIG. 1, in accordance with an embodiment of the invention. FIG. 2 is explained in conjunction with elements from FIG. 1. In FIG. 2, there is shown the block diagram 200 of the system 102. The system 102 includes at least one processor 202 (referred to as a processor 202, hereinafter), at least one non-transitory memory 204 (referred to as a memory 204, hereinafter), an input/output (I/O) interface 206, and a communication interface 208. The processor 202 is connected to the memory 204, the I/O interface 206, and the communication interface 208 through one or more wired or wireless connections. Although in FIG. 2, it is shown that the system 102 includes the processor 202, the memory 204, the I/O interface 206, and the communication interface 208, the present invention, however, may not be so limiting and the system 102 may include fewer or more components to perform the same or other functions of the system 102.

The processor 202 of the system 102 is configured to measure motion and recovery of the patient. The processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application-specific integrated circuit), an FPGA (field programmable gate array), a microcontroller unit (MCU), a hardware accelerator, a special-purpose computer chip, or the like. As such, in some embodiments, the processor 202 includes one or more processing cores configured to perform independently. A multi-core processor may enable multiprocessing within a single physical package. Additionally, or alternatively, the processor 202 may include one or more processors configured in tandem via the bus to enable independent execution of instructions, pipelining, and/or multithreading. Additionally, or alternatively, the processor 202 includes one or more processors capable of processing large volumes of workloads and operations to provide support for big data analysis. In an example embodiment, the processor 202 is in communication with the memory 204 via a bus for passing information among components of the system 102.

For example, when the processor 202 is embodied as an executor of software instructions, the instructions specifically configure the processor 202 to perform the algorithms and/or operations described herein when the instructions are executed. However, in some cases, the processor 202 may be a processor-specific device (for example, a mobile terminal or a fixed computing device) configured to employ an embodiment of the present disclosure by further configuration of the processor 202 by instructions for performing the algorithms and/or operations described herein. The processor 202 includes, among other things, a clock, an arithmetic logic unit (ALU), and logic gates configured to support the operation of the processor 202. The communication network 108 is accessed using the communication interface 208 of the system 102. The communication interface 208 provides an interface for accessing various features and data stored in the system 102.

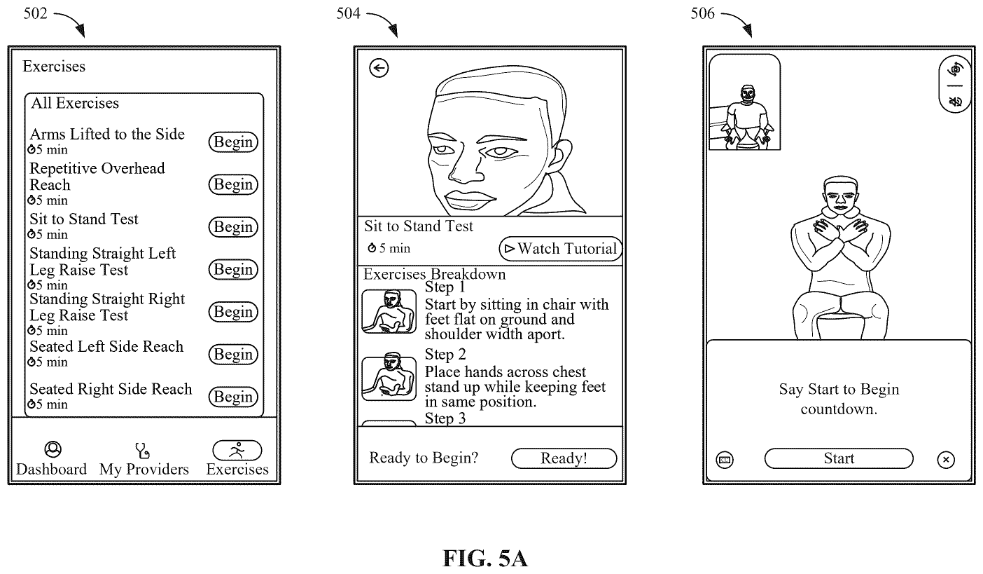

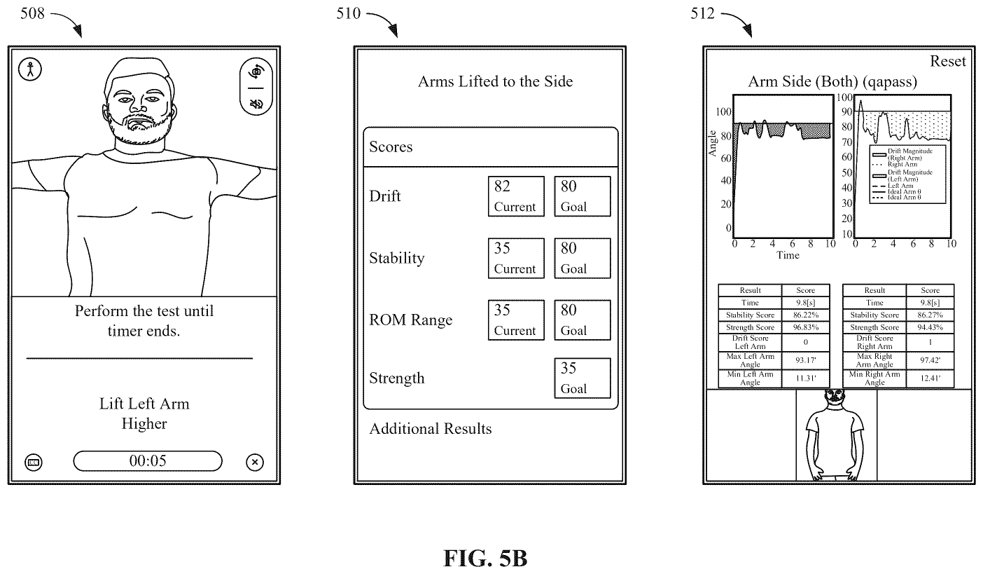

The functions or operations executed by the system 102, as described in FIG. 1, is performed by the processor 202. Operations executed by the processor 202 are described in detail, for example, in FIG. 3, FIG. 4, and FIGS. 5A and 5B.

The memory 204 may be non-transitory and may include, for example, one or more volatile and/or non-volatile memories. In other words, for example, the memory 204 may be an electronic storage device (for example, a computer-readable storage medium) comprising gates configured to store data (for example, bits) that are retrievable by a machine (for example, a computing device like the processor 202). The memory 204 is configured to store information, data, content, applications, instructions, or the like, for enabling the system 102 to carry out various functions in accordance with an example embodiment of the present invention. For example, the memory 204 is configured to buffer input data for processing by the processor 202. As exemplified in FIG. 2, the memory 204 is configured to store instructions for execution by the processor 202. As such, whether configured by hardware or software methods, or by a combination thereof, the processor 202 represents an entity (for example, physically embodied in circuitry) capable of performing operations according to an embodiment of the present invention while configured accordingly. Thus, for example, when the processor 202 is embodied as an Application Specific Integrated Circuit (ASIC), Field Programmable Gate Array (FPGA), or the like, the processor 202 is specifically configured hardware for conducting the operations described herein. In an embodiment, the memory 204 is configured to store a set of instructions 204a, and the AI model 110.

In an embodiment, the memory 204 is configured to store the user data and the anatomical data associated with the first user. In another embodiment, the processor 202 is configured to train the AI model 110 based on the user data and the anatomical data associated with the first user, and the generated score, and store the trained AI model 110 in the memory 204. In an exemplary embodiment, the AI model 110 is used for various tasks such as, but not limited to, classification, regression, pattern recognition, and decision-making.

In some example embodiments, the I/O interface 206 communicates with the system 102 and displays the input and/or output of the system 102. As such, the I/O interface 206 includes a display and, in some embodiments, also include a keyboard, a mouse, a touch screen, touch areas, soft keys, or other input/output mechanisms. In one embodiment, the system 102 includes a user interface circuitry configured to control at least some functions of one or more I/O interface elements such as a display and, in some embodiments, a plurality of speakers, a ringer, one or more microphones and/or the like. The processor 202 and/or I/O interface 206 circuitry including the processor 202 are configured to control one or more functions of one or more I/O interface 206 elements through computer program instructions (for example, software and/or firmware) stored on a memory 204 accessible to the processor 202.

The communication interface 208 includes the input interface and output interface for supporting communications to and from the system 102 or any other component with which the system 102 may communicate. The communication interface 208 may be any means such as a device or circuitry embodied in either hardware or a combination of hardware and software that is configured to receive and/or transmit data to/from a communications device in communication with the system 102. In this regard, the communication interface 208 includes, for example, an antenna (or multiple antennae) and supporting hardware and/or software for enabling communications with a wireless communication network. Additionally, or alternatively, the communication interface 208 includes the circuitry for interacting with the antenna(s) to cause transmission of signals via the antenna(s) or to handle receipt of signals received via the antenna(s). In some environments, the communication interface 208 may alternatively or additionally support wired communication. As such, for example, the communication interface 208 includes a communication modem and/or other hardware and/or software for supporting communication via cable, digital subscriber line (DSL), universal serial bus (USB), or other mechanisms.

Turning to FIG. 3, a diagram that illustrates exemplary operations for motion measurement and recovery of patients using artificial intelligence, in accordance with an embodiment of the invention is presented. Elements of FIG. 3 are explained in conjunction with elements from FIG. 1 and FIG. 2. With reference to FIG. 3, there is shown the diagram 300 of the system 102. The operations for motion measurement and recovery of patients are executed by any computing system, for example, by the system 102 of FIG. 1 or the processor 202 of FIG. 2. The operations may start at 302.

At 302, a user data is received. In yet another embodiment, the processor 202 is configured to receive the user data associated with a first user 304A. The user data includes, but is not limited to a demographic data 302A, a medication data 302B, and a historical data 302C.

The demographic data 302A corresponds to information associated with the first user 304A that describes a group of individuals. The demographic data 302A indicates prevalence of various orthopedic conditions associated with specific demography. Further, the demographic data 302A includes, but is not limited to age data, gender data, ethnicity data, and socioeconomic status. The age data corresponds to the age of the first user 304A. The age data indicates prevalence of various orthopedic conditions associated with specific age groups. For example, older adults may be more prone to osteoporosis. The gender data indicates prevalence of various orthopedic conditions associated with specific genders. For example, osteoporosis may be more common in postmenopausal women. The ethnicity data indicates prevalence of various orthopedic conditions associated with specific race or ethnicity due to genetic biases, geographical location, or environmental factors. The socioeconomic status indicates prevalence of various orthopedic conditions associated with various socioeconomic factors such as income, employment status or lifestyle.

Further, the medication data 302B corresponds to information associated with medical records of the patient. The medication data 302B includes but is not limited to information associated with medications being taken by the first user 304A, bone health data of the first user 304A, or pain areas associated with injuries. The historical data 302C corresponds to information associated with past medical records of the first user 304A. The historical data 302C includes but is not limited to information associated with past injuries suffered by the first user 304A, previous medications being taken by the first user 304A, chronic injuries, or previous therapeutic activities being suggested to the first user 304A.

In an example, the first user 304A creates an account on the online platform 104 to access the services of the online platform 104. For example, the first user 304A, such as an orthopedic patient, creates the account or logs in to a telemedicine application using credentials. Thereafter, the first user 304A is prompted to build a profile on the online platform 104, using an interactive web form available on the online platform 104. The interactive web form requires user data, such as, but not limited to, the demographic data 302A, the medication data 302B, and the historical data 302C, to build the profile. The system 102 is configured to receive user input indicative of personal information associated with the first user 304A. The personal information corresponds to the user data, such as but not limited to age, weight, height, or any medical condition associated with the first user 304A.

At 304, video data is received. In one embodiment, the processor 202 is configured to receive the video data indicative of an activity performed by the first user 304A. In another embodiment, the processor 202 is configured to control one or more image capturing devices 304B to capture the video data. In an example, the one or more image capturing devices 304B are communicatively coupled to the user device 106. In another example, the one or more image capturing devices 304B are integrated within the user device 106. The one or more image capturing devices 304B for example, includes image sensors, wide-angle cameras, digital cameras, 2D cameras, 3D depth cameras, RGB cameras, and combination thereof. In an example, the one or more image capturing devices are positioned strategically around the first user 304A to capture various angles and perspectives of the movement of the first user 304A. Further, the activity performed by the first user 304A corresponds to various exercises or movements performed by the first user 304A.

In an example, the first user 304A logs into the account on the online platform 104 to access the services of the online platform 104, using an application on the user device 106 such as a mobile phone. The first user 304A is prompted to build an anatomical profile on the online platform 104, using an interactive video form. For example, the first user 304A is instructed via the application on the user device 106 to keep the user device at a location and stand in front of the user device 106, such that a video data is captured using the camera associated with the user device 106. The user device 106 is positioned in a manner that the first user 304A is completely visible in the video data without any obstacles or hindrance.

In yet another embodiment, the processor 202 is configured to render a set of instructions associated with the activity to be performed by the first user 304A. In an example, the processor 202 is configured to render a set of instruction associated with a given set of exercises to determine the anatomical data associated with the first user 304A. Such functional assessment of the first user 304A allows the processor 202 to optimize the motion measurement. Further, the processor 202 is configured to render the set of instructions on a display screen associated with the user device 106. The given set of exercises includes but are not limited to an arm raise, an arm lean, a touch hands behind back, a stand up-sit down, a leg raise, and an arm curl. In an example, a first exercise of the given set of exercises corresponds to raising a right arm. The first user 304A is then instructed to raise the right arm up as far as possible, then down, and repeat 3 times. In another example, a second exercise of the given set of exercises corresponds to raising a left arm. The first user 304A is instructed to raise the left arm up as far as possible, then down, and repeat 3 times.

In an example, a third exercise of the given set of exercises corresponds to leaning a right arm. The first user 304A is instructed to stretch the right arm out to a side and then to lean right. In another example, a fourth exercise of the given set of exercises corresponds to leaning a left arm. The first user 304A is instructed to stretch the left arm out to a side and then to lean left.

In an example, a fifth exercise of the given set of exercises corresponds to touching hands behind the back. The first user 304A is instructed to attempt to touch both hands by first bringing the right arm up and behind across the back, and then bringing the left arm up and behind across the back. Alternatively, the first user 304A is instructed to attempt to touch both hands by first bringing the left arm up and behind across the back, and then bringing the right arm up and behind across the back. In another example, a sixth exercise of the given set of exercises corresponds to a stand up-sit down. The first user 304A is instructed to start in a sitting position, then to stand up and sit down and repeating 3 times.

In an example, a seventh exercise of the given set of exercises corresponds to a leg raise. The first user 304A is instructed to lift a right leg up as far up as possible, then lower the right leg back down. Alternatively, the first user 304A is instructed to lift a left leg up as far up as possible, then lower the left leg back down.

In an embodiment, the given set of exercises is stored in the memory 204, and the processor 202 is configured to receive a user input indictive of a selection of a given exercise from the given set of exercises to perform. The given set of exercises corresponds to a plurality of workouts associated with at least an upper body, a lower body, or a trunk of the first user 304A. The processor 202 selects the given exercise to be performed based on the user input and renders the set of instructions associated therewith. In another embodiment, the AI model 110 is further configured to select the given exercise to be performed based on the user data received.

At 306, a video data is processed. In an embodiment, the processor 202 is configured to process the video data. In an example, the video data includes a sequence of image frames. Each of the sequence of image frames is indicative of the first user 304A performing the given exercise. In an embodiment, the processing of the video data further includes a sequential execution of pre-processing operation, and marker-less motion capture operation on the video data. Details associated with the processing of the video data are provided, for example, in FIG. 4.

At 308, an anatomical data is determined. In an embodiment, the processor 202 is configured to determine the anatomical data associated with the first user 304A based on processing of the video data. The anatomical data includes but is not limited to a joints data for one or more anatomical joints of the first user 304A and a movement data associated with each of the one or more anatomical joints of the first user 304A. In an embodiment, the anatomical data further includes a limb parameter data associated with the first user 304A. In an embodiment, the joints data for the one or more anatomical joints of the first user 304A further includes but is not limited to a location data associated with each of the one or more anatomical joints, and an angle data associated with each of the one or more anatomical joints, as described for example, in FIG. 1.

In an embodiment, the movement data associated with each of the one or more anatomical joints of the first user 304A further includes but is not limited to a drift value associated with a movement of a first anatomical joint, an angular degree value associated with the movement of the first anatomical joint, a velocity value associated with the movement of the first anatomical joint, a strength value associated with the movement of the first anatomical joint, and a stability value associated with the movement data, as described for example, in FIG. 1.

In an embodiment, the processor 202 is configured to determine the anatomical data based on the set of instructions associated with the given set of exercises to be performed by the first user 304A. In an example, when the first user 304A performs the given exercise of the given set of exercises, the processor 202 is configured to receive the video data and analyze the video data to determine the anatomical data associated with the first user 304A in real-time. In an example, a first exercise of the given set of exercises corresponds to raising the right arm. The first user 304A is instructed to raise the right arm up as far as possible and then lower the right arm down. Further, the first user 304A is instructed to repeat the first exercise, for example, 3 times. Thereafter, the processor 202 is configured to analyze the video data indicative of the first user 304A performing the first exercise. The processor 202 is further configured to determine one or more metrics including, but not limited to, a number of repetitions, a maximum angle range of motion (ROM) in the right arm, and a stability of the first user 304A.

In another example, the fourth exercise of the given set of exercises corresponds to the leaning left arm. The first user 304A is instructed to stretch the left arm to out to the side and then lean to the left. Thereafter, the processor 202 is configured to analyze the video data indicative of the first user 304A performing the fourth exercise. The processor 202 is further configured to determine one or more metrics including, but not limited to, the stability of the arm, and a maximum angle of lean from a vertical axis.

In yet another example, the fifth exercise of the given set of exercises corresponds to touching hands behind the back. The first user 304A is instructed to attempt to touch both hands by bringing the right arm up and behind the back, and then to bring the left arm up, behind, and across the back. Thereafter, the processor 202 is configured to analyze the video data indicative of the first user 304A performing the fifth exercise. The processor 202 is further configured to determine one or more metrics including, but not limited to, the joint angles and ability to complete the fifth exercise as instructed.

In another example, the sixth exercise of the given set of exercises corresponds to the stand up-sit down. The first user 304A is instructed to start in a sitting position, then to stand up and to sit down again. Further, the first user 304A is instructed to repeat the sixth exercise, for example, 3 times. Thereafter, the processor 202 is configured to analyze the video data indicative of the first user 304A performing the sixth exercise. The processor 202 is further configured to determine one or more metrics including, but not limited to, a number of repetitions, and a stability of the movement.

In another example, the seventh exercise of the given set of exercises corresponds to the leg raise. The first user 304A is instructed to lift one leg up as far up as possible, then lower the leg back down, and to alternate this with both legs. Thereafter, the processor 202 is configured to analyze the video data indicative of the first user 304A performing the seventh exercise. The processor 202 is further configured to determine one or more metrics including, but not limited to, a maximum angle range of motion of hip and knee achieved by each leg.

At 310, a generated score is produced. In an embodiment, the processor 202 is configured to generate, using an artificial intelligence (AI) model 110, a generated score for the given exercise of the given set of exercises performed by the first user 304A based on the user data, and the anatomical data. In an embodiment, the generated score further includes a drift score associated with at least a first anatomical joint of the one or more anatomical joints, a stability score associated with at least the first anatomical joint of the one or more anatomical joints, a strength score associated with at least the first anatomical joint of the one or more anatomical joints, and a range score associated with at least the first anatomical joint of the one or more anatomical joints. The drift score associated with a movement of a first anatomical joint refers to a degree of deviation in the position of the joint over time due to prolonged or repetitive movements. Such deviation may occur due to muscle fatigue, ligament laxity, joint instability, or biomechanical imbalance. In other words, the drift score determines how far away the arm is over the course of the given exercise and maps that result to the drift score on a 0-4 scale.

The range score associated with the movement of the first anatomical joint refers to a maximum or a minimum angle value associated with the movement of the first anatomical joint. The strength score associated with the movement of the first anatomical joint refers to a force (or a torque) that joint produces or withstands during the movement. The strength score is associated with muscles, ligaments, tendons, and other supporting structures surrounding the joints. The stability score associated with the movement refers to the ability of the joint to maintain its position during various activities. Further, the stability score indicates the ability of the joint to maintain its position and resist displacement during the activity. For example, the stability score includes, but is not limited to, an inherent static stability of the joint and a dynamic stability of the joint during the movement. The joint with a high static stability may be less prone to dislocation even when at rest position. Further, the stability score accounts for the intermediary motion over the course of the given exercise. The stability score indicates a deviation from expected motion throughout the activity. Fundamentally, it quantifies that fact that it may be necessary to distinguish from the first user 304A holding the arms at a constant 80° as opposed to oscillating back and forth between 75° and 85° (which, depending on other calculations applied, can turn back similar scores).

In an example, the given exercise performed by the first user 304A corresponds to abduction, the first user 304A is instructed to stretch the arms out away from the torso. The generated score for the said exercise includes, but is not limited to, a maximum abduction degree (for example, maximum movement of the arm away from the body), a minimum abduction degree (for example, minimum movement of the arm away from the body), a stability score, a strength score, a total time (for example, a time taken to perform the activity), a drift score. For example, the drift score may correspond to a value of 0 indicative of no drift. Further the drift score of 0 indicates that a limb holds at 90 degrees or more for a full 10 seconds and drifts+/−10 degrees. The drift score may correspond to a value of 1 indicative of minimal drift. Further, the drift score of 1 indicates that the limb holds 90 degrees but drifts more than 10 degrees, thereby implying that the limb does not go down to 0 degrees and comes back up. The drift score may correspond to a value of 2 indicative of moderate drift. Further the drift score of 2 indicates that the limb may not stay at 90 degrees but may be able to maintain some effort against gravity, but eventually drops back down. The drift score may correspond to a value of 3 indicative of severe drift. Further the drift score of 3 indicates that the limb falls and there may be no effort against gravity. The drift score may correspond to a value of 4 indicative of no movement.

In an example, the first user 304A is instructed to lift the arms up. The generated score for the said exercise includes, but is not limited to, a maximum range of motion (for example, maximum shoulder angle degrees), a minimum range of motion (for example, minimum shoulder angle degrees), a stability score, a strength score, a total time (for example, a time taken to perform the activity), a drift score.

In an example, the first user 304A is instructed to stretch the arm out in front. The generated score for the said exercise includes, but is not limited to, a maximum range of motion (for example, maximum shoulder angle degrees), a minimum range of motion (for example, minimum shoulder angle degrees), a stability score, a strength score, a total time (for example, a time taken to perform the activity), a drift score.

In an example, the first user 304A is instructed to curl the arms. The generated score for the said exercise includes, but is not limited to, a maximum range of motion (for example, maximum elbow extension and a maximum elbow flexion degrees), a stability score, a strength score, a total time (for example, a time taken to perform the activity), a drift score.

In an embodiment, the user data may further include historical anatomical data associated with the first user 304A. The historical anatomical data corresponds to past anatomical data indicative of a past joints data for one or more anatomical joints of the first user 304A and past movement data associated with each of the one or more anatomical joints of the first user 304A. Thereafter, the processor 202 is configured to compare the historical anatomical data with the determined anatomical data. This allows determination of a deviation in the anatomical data of the first user 304A over a period of time. Such deviation may be indicative of a variation or an abnormality, while performing the given exercise. Further, the processor 202 is configured to generate the generated score for the given exercise performed by the first user 304A based on comparison thereof. Such a comparison between the historical anatomical data and the determined anatomical data provides insights associated with a recovery of the first user 304A.

In an embodiment, the processor 202 is configured to generate a report 310A associated with the first user 304A. The report 310A includes at least the user data, the anatomical data, and the generated score. With reference to FIG. 3, there is shown the report 310A indicative of the user data such as age, weight, height. Further, the report 310A includes a list of the given exercises performed by the first user 304A, such as, a left knee flexion, a right knee flexion, a left hip flexion and the like. Additionally, the report 310A includes the anatomical data and the generated score. In an embodiment, the processor 202 is configured to display the generated score on the online platform 104, thereby employing a visual representation of the generated score of the first user 304A to identify patterns or trends at a glance. Such visual representation may facilitate a user (such as medical professional) to determine a recovery level of the first user 304A based on the report 310A generated and make an informed decision as to the recovery level of the first user 304A. The generated score is displayed such as, but not limited to, a pie chart, a line chart, a bar chart, a histogram, a scatter plot, a radar chart, and the like.

The generated report further indicates distinct muscular requirements for controlling both motion and stability. In an example, the AI model 110 is trained to detect the alignment of limbs throughout multiple orientations and motions over time based on the anatomical data. The AI model 110 leverages basic kinematics, fundamental biomechanics, and signal processing to provide stable results for the first user 304A, that may be easily interpreted by medical professionals. In an example, each given exercise performed by the first user 304A stresses at least one different joint and the surrounding muscles. The AI model 110 identifies what is being tested, for example, is it a test intended to observe repetitions through a range of motion or does it assess the ability of a patient to maintain their limbs in a given orientation. Thereafter, based on the identification the score is generated using basic physics principles to calculate the strength needed for that motion (or lack of motion). For example, the first user 304A is instructed to hold their arms directly out to the front or the side at shoulder level. In such a case, the arms are fundamentally acting as rods hinged at the shoulder. Further, the processor 202 is configured to calculate strength based on concepts of rotational motion and net torques applied through the shoulder. On the contrary, while holding the hands above the head, the arm acts as a weight that is exerting a downward force on the body. Further, the processor 202 is configured to calculate the strength based on potential energy across height differences applied.

Additionally, the processor 202 is configured to employ data processing tools derived from signal processing to detect ranges of motion and repetitions during the given exercise being performed by the first user 304A. The range of motion alone may be employed by the medical professionals to assess flexibility and dexterity, and the repetitions, in a defined time period, to assess power and strength associated with the movement of the joint. In an example, the processor 202 is further configured to determine a maximum acceleration or force generated while performing the given exercise by the first user 304A. Additionally, the processor 202 is configured to determine a power generated by the legs over the course of a weighted squat by adding weight corrections.

In another embodiment, the patient may be instructed to perform Closed Kinetic Chain Unsupported Push-ups. In this given exercise, the first user 304A is instructed to a position having the first user 304A lay on their stomach, place palms flat on the floor, keep core tight, and push the body up toward the ceiling. Then the first user 304A is instructed to bend elbows to lower the body toward the ground, then push back up and straighten elbows while staying on the toes. The first user 304A is then instructed to attempt to repeat 10 times within 30 seconds. During such test, points of the body in the image frame may include waist, shoulder, head, elbow, wrist, hip, feet (for better visibility), and motion measurement. If the patient can perform 7-10 push-ups in 30 seconds, the generated score indicates good. On the contrary, if the patient performs 4-6 push-ups in 30 seconds, the generated score indicates fair, whereas, if the patient performs less than 4 push-ups, the generated score indicates poor.

FIG. 4 is a diagram that illustrates exemplary operations for marker-less motion measurement of users 304, in accordance with an embodiment of the disclosure. Elements of FIG. 4 are explained in conjunction with elements from FIG. 1, FIG. 2, and FIG. 3. With reference to FIG. 4, there is shown the diagram 400 of the system 102. The operations for motion measurement may be executed by any computing system, for example, by the system 102 of FIG. 1 or the processor 202 of FIG. 2. The operations start at 402.

At 402, the video data is received. In an embodiment, the processor 202 is configured to receive the video data indicative of the given exercise performed by the first user 304A, for example, as shown at 402A. In another embodiment, the processor 202 is configured to control an image capturing device to capture the video data. In an example, the image capturing device may be communicatively coupled to the user device 106. In another example, the image capturing device may be integrated within the user device 106. The image capturing device for example, a camera is employed to capture the video data indicative of the given exercise performed by the first user 304A. In yet another example, the camera is positioned strategically around the first user 304A to capture various angles and perspectives of the movement of the first user 304A. The given exercise performed by the first user 304A corresponds to various exercises or movements performed by the first user 304A. Examples of the image capturing device may include but are not limited to image sensors, wide-angle cameras, digital cameras, 2D cameras, 3D depth cameras, and RGB cameras.

At 404, the video data is processed. In another embodiment, the processor 202 is configured to process the video data. In an example, the video data includes a sequence of image frames. Each of the sequence of image frames is indicative of the first user 304A performing the given exercise. In yet another embodiment, the processing of the video data further includes a sequential execution of pre-processing operation, and marker-less motion capture operation on the video data.

In an example, the processor 202 is configured to determine a presence of the first user 304A within the video data including the sequence of image frames. Further, pre-processing operations are performed to enhance the image quality. Further, as illustrated in FIG. 4, the processor 202 is configured to identify a region of interest (such as the first user 402A) in the sequence of image frames, thereby adding a bounding box around the first user 402A. The processor 202 leverages use of the bounding box to visually indicate a position of the first user 402A. The processor 202 is configured to identify the first user 402A based on different image processing algorithms, such as, but not limited to, a face detection algorithms, an object detection algorithm, a deep learning algorithm, or other computer vison algorithms.

Based on the identified first user 402A, the processor 202 is configured to provide, as an input, a first image frame 404A, and a second image frame 404B to a pose detector module 404C. In an example, the processor 202 is configured to perform pre-processing operations on the sequence of image frames to enhance a quality of the video data. Such pre-processing operations include, but are not limited to, image resizing, noise reduction, and color correction. Thereafter, the pose detector module 404C employs a pose detection algorithm to determine a pose of the first user 404B. Examples of the pose detection algorithm include, but are not limited to, Blaze Pose algorithm, Pose Net algorithm, Open Pose algorithm, and Simple Baseline for human pose estimation.

In another embodiment, the processing of the video data includes processing of each image frame of the sequence of image frames to detect a pose of the first user 402A. The processor 202 is configured to process the sequence of image frames indicative of the first user 402A whose pose needs to be detected or tracked.

In yet another embodiment, the processor 202 is configured to employ the pose detection algorithm to determine one or more anatomical landmarks by pre-processing the video data. The anatomical landmarks correspond to anatomical key points such as, but not limited to, joints and body parts, including shoulders, elbows, wrists, hips, knees, and ankles. Further, the processor 202 is confirmed to detect, using the AI model 110, the pose of the first user 402A. The AI model 110 corresponds to a CNN model to perform the pose detection task efficiently. Upon determination of the anatomical points, the processor 202 is configured to perform pose alignment, thereby refining accuracy of the detected pose of the first user 402A. The pose alignment includes estimating spatial orientations between the determined anatomical key points, thereby ensuring consistency of the detected pose with human body biomechanics. Thereafter, the processor 202 is configured to control a pose tracker model 404D to perform pose tracking, thereby maintaining continuity in the detected poses of the first user 402A in real-time and near-real time.

In an embodiment, the processor 202 is configured to analyze the anatomical key points to determine the anatomical data, thereby accurately estimating the pose and movement of the first user 304A. The AI model 110 is trained to detect and track anatomical key points from the video data, thereby enabling estimating position on the fly.

In another embodiment, the AI model 110 is configured to analyze anatomical planes associated with the video data to estimate the pose of the human body. Examples of the anatomical planes include, but are not limited to, a coronal or frontal plane, a horizontal or axial or transverse plane, a sagittal or longitudinal plane. The frontal plane divides the body into front and back portions, thereby allowing to estimate movements away or towards the midline. The transverse plane divides the body into upper and lower portions, thereby allowing to estimate rotational movements. The sagittal plane divides the body into left and right portions, thereby allowing to estimate bending movements. The sagittal plane further includes a median plane or a mid-sagittal plane and a parasagittal plane. Such anatomical planes serve as reference points for determining orientation and movements of the human body. For example, the processor 202 is configured to estimate the pose of the first user 304A based on analysis of the anatomical key points relative to the anatomical planes, thereby optimally determining position, orientation, and movement of the body. The processor 202 leverages the use of the AI model 110 for motion capture by analyzing the anatomical key points relative to the anatomical planes.

At 406, marker-less motion data is determined. In an embodiment, the processor 202 is configured to determine marker-less motion data based on pre-processing of the video data. In an example, the processor 202 is configured to determine image data 406A based on pre-processing of the video data, a illustrated in FIG. 4. Such image data 406A correspond to a 2D color image and depth data. The processor 202 is configured to store the image data 406A in the memory 204. Thereafter, the processor 202 is further configured to digitize the image data 406A, and provide, as an input, the marker-less motion data to the AI model 110. The AI model 110 is further configured to analyze the marker-less motion data to determine the anatomical data.

Conventionally, in contrast, marker-based motion capture technology may be employed to track movement of the human body or estimate position of the human body. Such technology includes placement of sensors or reflective markers on specific anatomical landmarks of the human body to track movement or estimate position of the human body. Such sensors or reflective markers may reflect light emitted by cameras present in the vicinity of the human body, thereby allowing to reconstruct the 3-dimensional motion of the markers and by extension, the movement of the human body. Such marker-based motion capture technology may provide an accurate motion data, however, there are certain limitations associated therewith. For example, the cameras present in the vicinity of the user may have an unobstructed line of sight to the markers or sensors to prevent occlusion of the markers or sensors. Further, placement of markers or sensors on the human body requires expertise to obtain accurate and reliable motion tracking. Additionally, the placement of markers or sensors may restrict movement of the user, and cause discomfort to the user.

To overcome these challenges, the present invention provides a comprehensive method and system associated with marker-less motion capture technology that monitors the motion of the first user 402A in an efficient manner. The disclosed system 102 leverages the use of marker-less motion capture technology to accurately identify anatomical landmarks associated with the first user 402A, thereby optimally determining the anatomical data associated with the first user 402A. This allows the system 102 to be user friendly by improving technological ability to track patient movements without the need for sensors attached to patients' bodies via the user device 106.

At 408, one or more anatomical landmarks are identified. In an embodiment, the processor 202 is configured to identify one or more anatomical landmarks associated with the first user 402A based on the determined marker-less motion data. The processor 202 is configured to identify the one or more anatomical landmarks associated with the first user 402A based on the analysis of the marker-less motion data using the AI model 110. The one or more anatomical landmarks indicate anatomical topology of the first user 402A. Specifically, the anatomical landmarks correspond to a body topology including at least 33-points of anatomical key points topology, for example, as shown at 408A. Examples of the 33-points are associated with parts of the human body such as nose, left eye, right eye, left ear, right ear, left shoulder, right shoulder, left elbow, right elbow, mouth, left wrist, right wrist, fingers, hip, knee, ankle, heel, foot, etc.

In an example, the AI model 110 is configured to identify the one or more anatomical landmarks associated with the first user 402A, thereby performing a holistic landmarks detection that would be known to one ordinarily skilled in the art. Such anatomical landmarks include, but are not limited to, pose landmarks, hand landmarks, and face landmarks. The AI model 110 combines these landmarks to create a complete set of landmark points for the human body. This allows accurate determination of the movement of the human body. The identified anatomical landmarks are detected for each image frame and stored in the memory 204. In another example, a complete set of landmark points for the human body include a total of 543 anatomical key points including 33 anatomical key points associated with pose landmark detection, 42 anatomical key points associated with hand landmark detection (21 anatomical key points for each hand), and 468 anatomical key points associated with face landmark detection.

In yet another embodiment, the anatomical key points correspond to a 3-D geometry point including values of X, Y, and Z coordinates. As a pre-processing step, the presence of the human body is detected within an image frame using the pose detection model. Thereafter, a pose land-marker model is employed to estimate 33 3-D pose landmarks. Upon estimating the pose, the AI model 110 is configured to detect face landmarks and facial expressions in the video data. This allows identification of human facial expressions, application of facial filters and effects, and creation of virtual avatars. As a pre-processing step, the presence of human face within an image frame is detected using the face detection model. Thereafter, a face land-marker model is employed to estimate 478 3-D pose landmarks.

Once the one or more anatomical landmarks are identified, the processor 202 is configured to validate the landmarks to generate a new set of data called operational data. This operational data is then sent to the memory 204 for use by the next step, namely, the post-processing step, such as Analytics, to generate insightful data such as grades, scores, graphs, and charts.

At 410, anatomical data is determined. In an embodiment, the processor 202 is configured to determine the anatomical data associated with the first user 402A based on the identified one or more anatomical landmarks. The AI model 110 analyzes the anatomical landmarks to optimally determine anatomical data that includes joint kinematics data, for example, but not limited to, joint angles, joint positions, and limb parameters. Further, the marker-less motion data is analyzed to measure an angular degree of joint movement of the first user 304A.

Further, medical professionals utilize such joint kinematics to visualize variables associated with the joint movements, such as, but not limited to, joint velocities, accelerations, and torques to make informed decisions for the recovery of the patients. In an embodiment, the processor 202 is configured to determine a joint kinematics variable, using the AI model 110. The joint kinematics variable includes, but is not limited to, a velocity movement data, an acceleration movement data, and a kinetic forces movement data based on the marker-less motion capture data generating a calculated patient biometrics data.

In another embodiment, the motion data is mapped onto a digital skeleton or model to create a more intuitive representation of the body movement associated with the first user 304A. Additionally, the processor 202 is configured to refine the motion data using operations such as smoothing filtering or optimization, thereby removing noise, and improving accuracy of the motion data. This ensures natural looking movement associated with the captured motion data. The marker-less motion data is analyzed and visualized in several ways to gain insights into the movements, patterns, biomechanics, and performance associated with the first user 304A.

In an example, the processor 202 is configured to process the video data to identify anatomical key points such as joints, limbs, and other anatomical landmarks. The processor 202 is configured to employ a marker-less motion capture algorithm to analyze the video data. Further, the anatomical key points are detected based on color, texture, motion, depth, or combinations thereof. Based on the identified anatomical key topology the algorithm estimates the spatial configuration and pose of the first user 304A in each image frame of the video data. This involves reconstructing the 2D or 3D positions of the anatomical joints and segments typically represented as a skeleton model or set of connected anatomical key points to maintain temporal consistency and continuity in the motion capture data that is marker-less motion data. Further, the algorithm tracks the rejected anatomical key points across consecutive image frames of the video data, thereby allowing movement tracking overtime. Such tracking involves algorithms such as key point matching, motion prediction, temporal filtering, model-based tracking, and the like.

At 412, a score is generated. In an embodiment, the processor 202 is configured to compare the movement data associated with at least the first anatomical joint of the one or more anatomical joints and reference movement data associated with a corresponding anatomical joint of the one or more anatomical joints. Thereafter, the processor 202 is configured to generate the score for the given exercise performed by the first user based 304A on the comparison.

The AI model 110 is trained to determine distinct muscular requirements for controlling both motion and stability, thereafter, the processor 202 is configured to generate the score for the given exercise performed by the first user 304A based on the determination. In an example, the AI model 110 detects the alignment of limbs throughout multiple orientations and movements over time. Further, the processor 202 is configured to track performance of the first user 304A over time, thereby allowing comparison of the generated scores for the given exercises performed over time.

In an embodiment, the first user is instructed to perform seated lateral weight shift. In this exercise, the first user is instructed to raise one arm up to the side, while sitting in a chair. Thereafter, slowly lean while shifting weight from one buttock to the other while maintaining balance, hold for 10 seconds, and repeat on opposite side. During the test, the points of the body in the image frame include head, shoulder, trunk, knees, hips, ankles, elbows, and hands (for better visibility), and motion measurement. If the first user maintains the posture for 10 seconds or more, the generated score indicates good. On the contrary, if the first user maintains the posture between 5-10 seconds, the generated score indicates fair, whereas if the first user maintains the posture less than 5 seconds, the generated score indicates poor.

In another embodiment, the processor 202 is configured to determine a number of times the given exercise is performed by the first user based on the video data. The processor 202 is configured to determine a range of motion (ROM) associated with at least a first anatomical joint of the one or more anatomical joints based on the determined anatomical data. Thereafter, the processor 202 is configured to generate the score associated with at least the first anatomical joint for the given exercise performed by the first user based on the number of times the given exercise is performed and the range of motion associated therewith. In an example, if there is a modification in the number of times the given exercise is performed by the first user, and the range of motion (ROM) associated with at least the first anatomical joint of the one or more anatomical joints over time due to repetitions of the given exercise. The processor 202 is configured to adapt and update the generated score based on the modified data. In an example, the first user 304A is instructed to lean with the right arm out. The first user 304A repeats the given exercise 3 times for 10 seconds each, the drift score may correspond to 2, the stability score may correspond to 23.16 degrees, and the range of motion may correspond to 12.61-140.1 degrees. Therefore, the generated score would correspond to core range of motion indicative of 89.93-116.61 degrees.

In an example, if the number of repetitions correspond to 4-6 in 10 seconds, the generated score indicates fair. On the contrary if the number of repetitions corresponds to 7-10 in 10 seconds, the generated score indicates good, whereas if the number of repetitions corresponds to less than 3 in 10 seconds, the generated score indicates poor.

In another example, if the range of motion of the shoulder corresponds to 45-69 degrees for the duration of the given exercise, the generated score indicates fair. On the contrary if the range of motion of the shoulder corresponds to 70-100 degrees for the duration of the given exercise, the generated score indicates good, whereas if the user is unable to maintain the shoulder position for the duration of the given exercise, the generated score indicates poor. Thereafter, the system 102 is configured to provide suggestions to modify the movement or postures based on the generated score, thereby optimizing the recovery process.

In an embodiment, the processor 202 is configured to detect a facial region of the first user based on the processing of the video data. The processor 202 is configured to determine one or more facial features associated with the first user based on the detected facial region. Thereafter, the processor 202 compares at least the first feature from the one or more facial features and a corresponding reference facial feature from one or more reference facial features and renders the set of instructions associated with the given exercise to be performed by the first user based on the comparison. In an example, the processor 202 is configured to detect the facial region of the first user in the received video data based on the image processing algorithm, such as, but not limited to, face detection methods, object detection methods, deep learning methods, and other image processing methods. Based on the detected facial regions, the processor 202 is configured to track the head position of the first user. Further, the AI model 110 employs a pre-trained deep learning model for example, including but not limited to OpenCV to detect and analyze facial expression in the video data. The AI model 110 is employed to extract facial landmarks from each image frame of the video data. Such facial landmarks include for example, not limited to facial expressions, head movements, eye movements, eyebrow positions, mouth shape, and the like. Thereafter, the processor 202 compares at least the first feature from the one or more facial features and a corresponding reference facial feature from one or more reference facial features and renders the set of instructions associated with the given exercise to be performed by the first user based on the comparison. For example, if the first user suffers from facial paralysis due to stroke, thereby leading to eyebrow drooping, eye closure, mouth asymmetry, facial drooping, and the like. The processor 202 is configured to compare the facial features of the first user 304A with a corresponding reference facial feature to determine the deviation. Based on the deviation, the AI model 110 suggests the given exercises to be performed by the first user 304A to improve the corresponding facial feature.

In an embodiment, the proposed invention corresponds to a smart-phone compatible marker-less motion capture (MMC) technology that allows a telemedicine pipeline among patients (such as, the first user) and medical professionals. The system 102 uses a commercially available smartphone to track, assess, and recommends improvements to patient recovery and treatment plans for stroke victims and potentially other ailments. The system 102 obtains continuous data regarding patient recovery and rehabilitation plans, thereby providing optimized treatment and rehabilitation plans based on the individual patient. Thus, improving technological ability to track patient movements and rehabilitation progress via patients' personal portable electronic devices. The system 102 limits the need for attaching sensors to patients' bodies for the development of improved rehabilitation and treatment plans for the individual patient. Further, the system 102 leverages the use of AI to determine the most effective treatment plans for patient recovery compared to demographic information. Therefore, assessing each patient's recovery progress and effectiveness of treatment plans and aggregates the data collected in the assessment, and continues to collect such data over time.

Further, the system 102 provides the anatomical data (such as joint kinematics data), leading to accurate description of motion geometry and informing relevant medical attributes. The system 102 provides real-time assistance, personalized adjustments to treatment and recovery plans, resulting in significantly decreased costs due to not needing additional sensors, continuous data collection with significantly increased frequency for research and optimization of treatment and recovery plans for similar patients over time. This provides optimum suggestions for the personalized treatment plans, and medications to improve the recovery process of the patient.

FIG. 5A and FIG. 5B are diagrams that illustrate exemplary user interfaces for measurement and recovery of the patients, in accordance with an embodiment of the invention. Elements of FIG. 5A and FIG. 5B are explained in conjunction with elements from FIG. 1, FIG. 2, FIG. 3, and FIG. 4. With reference to FIG. 5A, and FIG. 5B, there are shown exemplary user interfaces for measurement and recovery of the patients.

The user interface 502 displays a list of exercises being performed by the first user 304A. Further, the user interface 502 includes user data. In operation the first user 304A may select a first given exercise from the list to be performed. Thereafter, the processor 202 is configured to render the set of instructions associated with the selected first given exercise.

In an example, the user interface 502 displays exercises associated with upper extremities or lower body of the first user 304A. The exercises associated with the upper extremities may include activities such as, but not limited to, Bilateral AROM shoulder abduction, static hold (commonly known as arms lifted to the side), and Bilateral AROM shoulder flexion, repetition (commonly known as repetitive overhead reach). Further, the exercises associated with the lower body may include, but are not limited to, sit-and-stand test, and single stance straight leg raise stability test.

In an example, the first user 304A has access to a list of exercises involving the upper body, lower body, and trunk. The first user 304A may select assessment or exercise mode for each exercise. Further, the user device 106 is able to be interactive and can count repetitions and give recommendations on techniques (e.g. raise your arm higher, hold for 5 seconds etc.).

In an embodiment, the processor 202 is configured to receive a user input associated with the first user indicative of a mode in which the given exercise is to be performed. Examples of the mode include, exercise mode and assessment mode. In the exercise mode, the first user 304A selects a given set of exercises to be performed. In an example, the given exercise is generated by the AI model 110 based on the user data. In another example, the medical professional suggests or assigns the given exercise to be performed based on the physical analysis of the user data. Further, in the assessment mode, the first user 304A performs a given list of exercises for evaluation, and the processor 202 is configured to generate the score based on the given exercise being performed to assess motion stability and strength of the user.

In an example, for each given exercise, there may be a silent mode and a voice mode. The silent mode has a live description of what is to be done, as well as writing. In other words, the instructions are displayed on the user device 106 and an avatar performs the given exercise along with the first user 304A. Further, there might be a button for skipping the description if needed. Once the patient is in frame, the border of the screen turns green, and then the countdown starts. Alternatively, there will also be a button or question to determine whether the first user 304A is ready to perform the given exercise. On the contrary, in the voice mode the instructions are spoken out loud for the first user 304A to adhere to, in addition to the written instruction, and the description of what is to be done. Further, a countdown timer is present before the start of the given exercise. In an embodiment, during live assessment, there is a glowing button on the screen to depict live assessment to indicate live evaluation.

The user interface further includes display of a short description associated with the selected given exercise, along with additional instructions. In an example, the description corresponds to “lift both arms to shoulder level, thumbs up, hold for 10 seconds”, “Standing with arms by your side, thumbs pointing up, actively raise your arms straight in front of you and overhead. Repeat as many times as you can in 10 seconds”, “From a seated position, stand, then return to sitting, while keeping your feet in the same position. Repeat 5 times”, and “While standing next to a chair for support, lift one leg forward and off ground with a straight knee as high as you can. Repeat on opposite leg”. Further the additional instructions include text indication such as “Face the camera, feet shoulder width apart, have arms by your side with thumbs pointing out and up. When test starts, raise arms up and out by your sides to shoulder level and hold for 10 seconds”, “Start by sitting in chair with feet flat on ground and shoulder width apart. Place hands across chest, stand up while keeping feet in same position. Complete 5 stands as quickly and safely as you can. The timer will stop after you sit from your 5th stand”, and “While standing next to a chair for support, lift one leg forward off the ground with a straight knee as high as you can. Hold for 10 seconds. Return to starting position and repeat with opposite leg.”

For example, as shown in a user interface 504, the first user 304A may have selected sit to stand exercise, the user interface 504 displays the set of instructions associated with the exercise, for example, “start by sitting in chair”, “place hands across chest” and the like.

In another example, user input is required to select or start the activity. In an embodiment, the processor 202 is configured to receive the user input including, not limited to, a text input, a dropdown menu, slider options, and the like. In yet another example, the user triggers action by clicking buttons like “Ready”, “Submit”, or “Cancel”. In even yet another example, the user input corresponds to a voice input including instructions such as, but not limited to, “start” and “stop” to start and stop the given exercise, respectively.

As shown in user interface 506, the first user 304A is instructed to provide the voice input to start the given exercise. Further, the user interface 506 includes a small box indicative of the video data being received and an avatar representation. In an embodiment, the processor 202 is configured to generate an avatar based on the received video data. In another embodiment, the processor 202 is configured to receive user input indicative of a selection of avatars from a list of available options. Such an avatar may mimic a visual representation of the given exercise being performed by the first user in real-time. Such visualization may enable the user to accurately visualize the motion being performed and correct the movement based on the visualization.

In an embodiment, the processor 202 is configured to determine the anatomical data based on the set of instructions. Further, the processor 202 is configured to generate an output indicative of a modification of a movement of at least the first anatomical joint of the one or more anatomical joints based on the generated score. The modification of the movement is indicative of the correction or adjustment of the current movement to reach optimum level. In an example, the AI model 110 is configured to provide suggestions to modify the movement to obtain a desired result. In another example, the processor 202 is configured to transmit the generated score to a second user (for example the medical professional). The second user analyzes the video data and the generated score to assess the motion stability and the strength of the first user, thereafter, providing a user input indicative of an update to the generated output. Such an update is indicative of the suggestions to adjust or correct the movement to obtain the desired result.

FIG. 5B, illustrates the user interface 508 indicating a user performing a given exercise to form a T-shape. The processor 202 is configured to analyze the video data received and provide suggestions for example, but not limited to, lifting left arm higher to correct the movement.

A user interface 510 displays the generated score for the given exercise being performed and a reference score (or a fitness goal). The reference score is determined by the AI model 110 based on the user data and the anatomical data. Further, a user interface 512 displays the report generated.

In an embodiment, the processor 202 is configured to receive a user input associated with the first user and modify the set of instructions associated with the given exercise to be performed by the first user based on the received user input. In another embodiment, the first user provides input indicative of modification in the activity. The processor 202 is configured to modify the given exercise based on the received user input. The modification to the given exercise corresponds to, but not limited to, adjusting repetition, time of holds, duration of the given exercise. In another embodiment, the user modifies the given exercise based on the generated score to improve the stability and strength of the motion. Further, the processor 202 is configured to update the reference score based on the modification by the first user. For example, if current scoring is based on number of repetitions in 10 seconds and the good score would be 7-10 repetitions then, if the first user customized the given exercise and made it for 60 seconds then the scoring would be adjusted accordingly.

The proposed system 102 utilizes the marker-less motion capture (MMC) technology. The system 102 directs the patient (the first user 304A) to perform certain movements (given exercise) after confirming the presence of the patient in the image frame of the camera by displaying a green box around the border of the smartphone (user device 106). Thereafter, the patient is then directed to perform certain movements that are captured by the camera and are then uploaded to a remote cloud server where the AI model 110 calculates movement angles and identifies the joint locations, as well as, the movement of the patient. Further, the AI model 110 generates an Augmented Reality video of the patient superimposing the movement angles, joint points, and the like. Additionally, the AI model 110 evaluates and correlates the video data with biometric, historical, and normal patient database to generate the score. This data is then accessible by a medical provider (such as the second user) through a custom provider web portal (such as the online platform) where the medical provider can review the data and the movement videos while providing adjustments and assessments of the patient's treatment plan along with suggestions generated by the AI model 110. In another embodiment, the system 102 leverages the use of AI model 110 to assess in real-time the patient's movements and direct the patient to make adjustments or corrections to the movements. In yet another embodiment, the AI model 110 generates a report associated with the first user. The processor 202 is configured to determine a recovery level of the first user based on the generated report. The system 102 is configured to customize the given exercise to be performed and reference score by adjusting the variables above and that any of the exercises could be pulled into a customizable assessment as well based on the generated report, and the recovery level of the patient.

In an example, the patient is asked to raise both arms all the way up and then back down. Further, this is to be repeated for a total of 30 times. This may be either 3 sets of 10, or 30 times. The objective is to have the patient do as many repetitions as possible in 30 seconds. Further, a difficult level involves the patient raising their arms up and then holding at that position for 3 seconds then lowering back down. The user device 106 will display the instruction to the patient to hold the arms up and then lower the arms down. Each layer of difficulty will make the patient hold up for 3 more seconds (e.g. 6 seconds, 9 seconds and the like). In another example, the patient holds both arms up for 30 seconds while a countdown is done. There is an option for doing one arm at a time. Additionally, there is another option for alternating arms. Moreover, the user device 106 should have an “additive” beep/sound with each rep as well as a ticking number with each rep. The system 102 will measure the maximum angle of the arm to trunk at maximum raise. Further, it will also measure the speed of raising of the arm from rest to maximum, steadiness of the arm during this given exercise (e.g. unsteady, moderate, optimal), and a number of reps that were done in 30 seconds.

In an embodiment, the system 102 provides suggestions to optimize recovery of the patient such as, but not limited to, “hold posture for 10 seconds”, “raise right arm”, “raise left arm”, “do not shrug shoulders”, “continue to repeat overhead movement”, “Keeps arms crossed”, and the like. Such a system may be employed to overcome the limitations due to a shortage of healthcare professionals, and economic constraints that may limit a number of therapy sessions covered by insurance. Therefore, the proposed system 102 achieves further optimal outcomes in addition to patient recovery.

In another example, for the arm/leg test, the system 102 measures the angle of extremity to the trunk (body's vertical line). In such a case, the arm at side is 0 degrees, and the arm raised straight up is 180 degrees. Further, the system 102 measures the angle of extremity to the body's horizontal axis (shoulder to shoulder line, hip to hip), extremity to the horizontal axis of the space, extremity to the vertical axis of the space, length of full extremity (upper and lower), length of parts of extremity (shoulder to elbow, elbow to wrist, wrist to fingertips if possible, more important shoulder to elbow and elbow to wrist), angle of elbow joint (forearm to arm), angle of knee joint (leg to thigh).

In yet another example, for the arm raised forward test, the patient is instructed to raise arms forward and then to rest. Additionally, for the arms raised outward (abduction), the patient is instructed to raise arms outwards and then to rest.

In another example, in the push your hands forward test, the patient is instructed to push the hands forward and then back to starting position. The system 102 measures the speed of reps in 30 seconds (when applies). Further, the system 102 measures the angle of the elbow joint (forearm to arm) at the maximal position. In an ideal scenario the angle of the elbow joint is closer to 180 degrees. The system 102 measures the steadiness of the extremity in this process.

In yet another example, in the Stretch/fan arms out, the patient is instructed to fan arms outward and then back to starting position. The system 102 measures the angle of the arm to trunk. In the rest position that angle should ideally be zero. Further, the system 102 measures the speed of reps in 30 seconds (when applicable). The system 102 will measure the angle of the elbow joint (forearm to arm) at the maximal position that would be closer to 180 degrees in an ideal scenario. The system 102 will measure steadiness of the extremities during the test.

In the arm raise leaning test, the patient is asked to alternate raising each arm in that position and then back to rest. The system 102 measures the steadiness of the patient and the speed at which they achieve this. Further, in the shoulder shrug test, the patient is asked to shrug both shoulders and then go back to rest position. The system 102 measures the level of shrug (shoulder tip to base of anterior neck as a line over the transverse line of the base of the anterior neck).

In the Sit-to-stand test, the patient is asked to stand up from sitting. The system 102 measures the number of reps in 30 seconds. In an example, if the patient is not able to perform the given exercise on their own, a helper may be provided. Further, in the hip flexion test, the patient is asked to flex the hip (by raising the knee). The system 102 measures the reps in 30 seconds. The system 102 measures the maximum angle of flexion from thigh to trunk. Further, the system 102 measures steadiness of the motion.

In the knee extension, the patient is asked to extend the knee (Kick forward). The system 102 measures the angle at the knee joint, a maximum 180 degrees ideally. Further, a degree of difficulty may be increased based on increasing the time to hold knee at extended position in increments of 3 seconds. Further, in the plantar flexion test, the patient is asked to stretch out the leg and extend the ankle (like pressing on a gas pedal). The device 106 will measure the angle at the ankle. In the outstretch the leg test, the patient is asked to outwardly stretch the leg. Further, in the curl test, the patient is asked to flex the elbow and then return to rest. The patient will do this repeatedly during the time they are being instructed.

In an embodiment, the AI model 110 is configured to perform posture matching tasks. Initially, the patient may be at rest. While at rest, the system 102 captures the video data indicative of the patient's rested posture. The system 102 then poses a silhouette of an extremity (upper or lower) posture based on the patient, and the patient is to superimpose their extremity onto it. The silhouette pose of the extremity will have a continuous blinking red light to insinuate that the patient needs to superimpose their extremity on it. Once the patient superimposes their extremity in it, there is a “rewarding” sound that goes off. The patient is then given another posture to simulate, and so on. The patient can be given one minute to complete postures and see how many can be done in that minute. The difficulty can be based on how big or small the silhouette is; the silhouette can also move, and the patient is instructed to follow it.

In an embodiment, the system 102 renders a ball at a distance and the ball starts to approach the screen. Once the ball fills a certain part of the screen associated with the user device 106, the patient is instructed to swat the ball into the screen. Then the ball goes back into the screen and the cycle starts over. The objective is to get the patient to utilize their extremity and use coordination to time when to use the extremity. Further, difficulty can vary based on speed of the rotation of the ball.

In another embodiment, the system 102 renders a certain color on the screen. Further, the system 102 receives the touch input from the patient. Based on the touch input, the system 102 automatically recognizes the patient's hand/s and simulates the hands as an eraser or marker. The patient then imitates clearing away the old color to make a completely new color. This could be timed or not timed. The difficulty could be based on the size of the eraser/marker to the screen, also from the task being timed.

The disclosed system 102 is further employed to utilize current Telehealth medical AI technology to address employers' concerns regarding work specific testing. The aim is to provide accurate and reliable fit-for-duty assessments for return-to work evaluations. Further, the disclosed system 102 is employed to make better job specific matches and provides accurate return to work recommendations. For example, the processor 202 is configured to receive a user input indicative of an assessment associated with fit-for duty report. The user performs the activities for the assessment, thereafter the processor 202 is configured to generate the score for the activity being performed. Once the assessment is complete, the results are stored in the memory 204 and the processor 202 is configured to validate and analyze the results. This provides Forensic recording, developing test integrity, displaying client consistency of effort and reliability of client reports for Pre/Post offer screenings, Functional Capacity Evaluations, and Impairment Ratings. The system 102 facilitates analysis and ensures accurate capturing of impairments, thereby providing a comprehensive record of the user fitness. This may be beneficial to accommodate for individuals' disability and guide through rehabilitation.

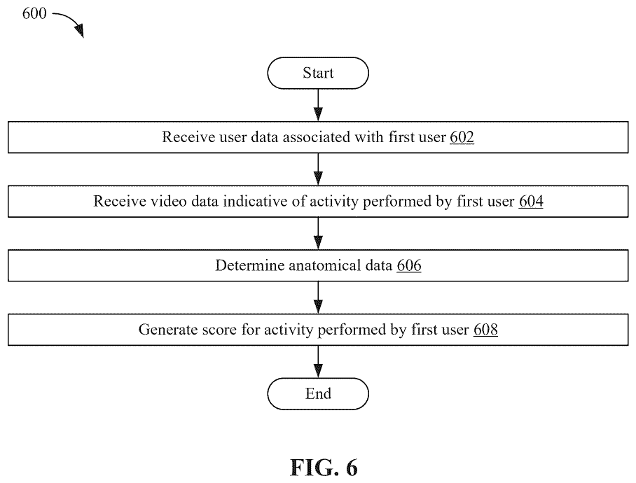

FIG. 6 is a flowchart 600 that illustrates an exemplary method for motion measurement and recovery of the patients using artificial intelligence, in accordance with an embodiment of the invention. FIG. 6 is explained in conjunction with elements from FIGS. 1, 2, 3, 4, 5A, and 5B. With reference to FIG. 6, there is shown flowchart 600. The operations of the exemplary method are executed by any computing system, for example, by the system 102 of FIG. 1 or the processor 202 of FIG. 2. The operations of the flowchart 600 start at 602.

At 602, user data is received. In an embodiment, the processor 202 is configured to receive the user data associated with a first user. The user data includes demographic data, medication data, and historical data. Details associated with the user data are provided for example, in FIG. 3.

At 604, video data is received. In an embodiment, the processor 202 is configured to receive the video data indicative of an activity performed by the first user. Details associated with the reception of video data are provided, for example, in FIG. 3 and FIG. 4.

At 606, anatomical data is determined. In an embodiment, the processor 202 is configured to determine the anatomical data associated with the first user based on processing of the video data. The anatomical data includes joints data for one or more anatomical joints of the first user and movement data associated with each of the one or more anatomical joints of the first user. Details associated with the determination of the anatomical data are provided, for example, in FIG. 3 and FIG. 4.

At 608, a score is generated. In an embodiment, the processor 202 is configured to generate, using an artificial intelligence (AI) model 110, the score for the activity performed by the first user based on the user data, and the anatomical data. Details associated with the generation of the score are provided, for example, in FIG. 3 and FIG. 4.

Alternatively, system 102 comprises means for performing each of the operations described above. In this regard, according to an example embodiment, examples of means for performing operations may comprise, for example, the processor and/or a device or circuit for executing instructions or executing an algorithm for processing information as described above.

Various embodiments of the invention provide a non-transitory computer-readable medium and/or storage medium having stored thereon, instructions executable by a machine and/or a computer to operate a system (e.g., the system 102) for motion measurement and recovery of the patients using artificial intelligence. The instructions cause the machine and/or computer to perform operations including receiving user data associated with a first user. The user data includes demographic data, medication data, and historical data. The operations further include receiving video data indicative of an activity performed by the first user. The operations further include determining anatomical data associated with the first user based on processing of the video data. The anatomical data includes joints data for one or more anatomical joints of the first user and movement data associated with each of the one or more anatomical joints of the first user. The operations further include generating, using an artificial intelligence (AI) model 110, a score for the activity performed by the first user based on the user data and the anatomical data.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of reactants and/or functions, it should be appreciated that different combinations of reactants and/or functions may be provided by alternative embodiments without departing from the scope of the appended claims. In this regard, for example, different combinations of reactants and/or functions than those explicitly described above are also contemplated as may be set forth in some of the appended claims. Although specific terms are employed herein, they are used in a generic and descriptive sense only and not for purposes of limitation.

The claimed invention is:

1. A system, comprising:
one or more processors; and
a memory coupled to the one or more processors, the memory having stored therein instructions executable by the one or more processors to configure the system to:
receive a user data associated with a first user, wherein the user data comprises a demographic data, a medication data, and a historical data;
receive a video data indicative of an activity performed by the first user;
determine an anatomical data associated with the first user based on processing of the video data, wherein the anatomical data comprises: a joints data for one or more anatomical joints of the first user and a movement data associated with each of the one or more anatomical joints of the first user; and
generate, using an artificial intelligence (AI) model, a score for the activity performed by the first user based on the user data, and the anatomical data.

2. The system of claim 1, wherein the joints data for the one or more anatomical joints of the first user further comprises: a location data associated with each of the one or more anatomical joints, and an angle data associated with each of the one or more anatomical joints.

3. The system of claim 1, wherein the movement data associated with each of the one or more anatomical joints of the first user further comprises: a drift value associated with a movement of a first anatomical joint, an angular degree value associated with the movement of the first anatomical joint, a velocity value associated with the movement of the first anatomical joint, a strength value associated with the movement of the first anatomical joint and a stability value associated with the movement.

4. The system of claim 1, wherein the one or more processors are further configured to:
determine a marker-less motion data based on the processing of the video data;
identify one or more anatomical landmarks associated with the first user based on the marker-less motion data; and
determine the anatomical data based on the identified one or more anatomical landmarks associated with the first user.

5. The system of claim 1, wherein the anatomical data further comprises a limb parameter data associated with the first user.

6. The system of claim 1, wherein the one or more processors are further configured to:
render a set of instructions associated with the activity to be performed by the first user; and
determine the anatomical data based on the set of instructions.

7. The system of claim 6, wherein the one or more processors are further configured to:
receive a user input associated with the first user; and
modify the set of instructions associated with the activity to be performed by the first user based on the received user input.

8. The system of claim 6, wherein the AI model is further configured to generate the set of instructions based on the received user input.

9. The system of claim 1, wherein the one or more processors are further configured to generate an output indicative of a modification of a movement of at least the first anatomical joint of the one or more anatomical joints based on the generated score.

10. The system of claim 1, wherein the one or more processors are further configured to:
transmit the generated score to a second user;
receive a user input associated with the second user; and
update the generated output based on the user input associated with the second user received.

11. The system of claim 1, wherein the one or more processors are further configured to:
generate a report associated with the first user, wherein the report comprises at least: the user data, the anatomical data, and the generated score; and
determine a recovery level of the first user based on the generated report.

12. The system of claim 1, wherein the one or more processors are further configured to:
receive at least a first image of an environment from an image capturing device;
determine a presence of the first user in the environment based on the received first image;
render the activity to be performed by the first user; and
obtain the video data associated with the rendered activity to be performed by the first user.

13. The system of claim 1, wherein the one or more processors are further configured to:
determine a number of times the activity is performed by the first user based on the video data;
determine a range of motion associated with at least a first anatomical joint of the one or more anatomical joints based on the determined anatomical data; and
generate the score associated with at least the first anatomical joint for the activity performed by the first user based on the number of times the activity is performed and the range of motion.

14. The system of claim 1, wherein the generated score further comprises: a drift score associated with at least a first anatomical joint of the one or more anatomical joints, a stability score associated with at least the first anatomical joint of the one or more anatomical joints, a strength score associated with at least the first anatomical joint of the one or more anatomical joints, and a range score associated with at least the first anatomical joint of the one or more anatomical joints.

15. The system of claim 1, wherein the one or more processors are further configured to:
detect a facial region of the first user based on the processing of the video data;
determine one or more facial features associated with the first user based on the detected facial region;
compare at least a first feature from the one or more facial features and a corresponding reference facial feature from one or more reference facial features; and
render a set of instructions associated with the activity to be performed by the first user based on the comparison.

16. The system of claim 1, wherein the user data further comprises historical anatomical data associated with the first user, and wherein the one or more processors are further configured to:
compare the historical anatomical data with the determined anatomical data; and
generate the score for the activity performed by the first user based on the comparison.

17. A system, comprising:
one or more processors; and
a memory coupled to the one or more processors, the memory having stored therein instructions executable by the one or more processors to configure the system to:
receive a video data indicative of an activity performed by a first user, wherein the video data comprises a sequence of images;
determine an anatomical data associated with the first user based on the processing of the video data, wherein the anatomical data comprises: a joints data for one or more anatomical joints of the first user, and a movement data associated with each of the one or more anatomical joints of the first user;
compare the anatomical data associated with the first user and a historical anatomical data; and
generate a score associated with the activity performed by the first user based on the comparison.

18. The system of claim 17, wherein the one or more processors are further configured to:
determine a marker-less motion data based on the processing of the video data;
identify one or more anatomical landmarks associated with the first user based on the marker-less motion data; and
determine the anatomical data based on the identified one or more anatomical landmarks associated with the first user.

19. A method, comprising:
receiving a user data associated with a first user, wherein the user data comprises a demographic data, a medication data, and a historical data;
receiving a video data indicative of an activity performed by the first user;
determining an anatomical data associated with the first user based on the processing of the video data, wherein the anatomical data comprises: a joints data for one or more anatomical joints of the first user, and a movement data associated with each of the one or more anatomical joints of the first user; and
generating, using an AI model, a score for the activity performed by the first user based on the user data, and the anatomical data.

20. The method of claim 19, further comprising:
determining a marker-less motion data based on the processing of the video data;
identifying one or more anatomical landmarks associated with the first user based on the marker-less motion data; and
determining the anatomical data based on the identified one or more anatomical landmarks associated with the first user.
